(19) United States
(12) Patent Application Publication
(10) Pub. No.: US 2023/0406805 A1
(43) Pub. Date: Dec. 21, 2023

(54) PROCESS FOR PRODUCTION OF ACETIC ACID AND ACRYLIC ACID FROM WASTE CARBON CONTAINING MATERIALS WITH REDUCED CARBON FOOTPRINT

(71) Applicant: ENERKEM INC., Montreal (CA)

(72) Inventors: David LYNCH, Beaumont (CA); Prashant KUMAR, Edmonton (CA); Imtiaz AHMED, Edmonton (CA)

(21) Appl. No.: 18/253,619

(22) PCT Filed: Nov. 4, 2021
(86) PCT No.: PCT/CA2021/051565

§ 371 Date: May 19, 2023

Related U.S. Application Data
(60) Provisional application No. 63/118,103, filed on Nov. 25, 2020.

(51) Int. Cl. C07C 51/373

(52) U.S. Cl. CPC C07C 51/373; C07C 2527/167

(57) ABSTRACT

It is provided a process of converting syngas resulting from the gasification of a carbonaceous material into acetic acid and acrylic acid comprising converting the syngas into methanol and separating the methanol into a first and second stream, carbonylation of the first stream of methanol producing methyl acetate, hydrolyzing the methyl acetate to obtain acetic acid, oxidizing the second stream of the methanol into formaldehyde in a gas phase reaction, and reacting by aldol condensation the formaldehyde and acetic acid to produce acrylic acid. Particularly, the first stream of methanol is dehydrated to produce dimethyl ether (DME) and the DME is further contacted with syngas under an iodide-free environment to produce the methyl acetate by carbonylation, and subsequently acetic acid using a reactive distillation column.

### CROSS-REFERENCE TO RELATED APPLICATION

The present application is claiming priority from U.S. Provisional Application No. 63/118,103 filed Nov. 25, 2021, the content of which is hereby incorporated by reference in its entirety.

### TECHNICAL FIELD

It is provided a process of converting syngas into acetic acid and/or acrylic acid via methanol production.

### BACKGROUND

Acrylic acid (AA) is a valuable chemical industry product. The main application of AA and its derivatives is production of various polymer materials, super absorbents, paint-and-varnish materials etc. The global production jumped from 1.3 million metric tons in 2000 to 5.0 million metric tons in 2015 and is expected to grow to 7.2 million metric tons by 2023. Although acrylic acid is predominantly used as a raw material for acrylic esters, a trend in the industry is the rising demand for superabsorbent polymers. Accounting for about 33% of the global acrylic acid supply in 2016, it has experienced very strong growth primarily in the personal disposable hygiene products such as baby diapers, adult protective underwear and sanitary napkins.

As petrochemical resources become increasingly scarce, more expensive, and subject to regulations for CO₂ emissions, there exists a growing need for bio-based acrylic acid, acrylic acid derivatives, or mixtures thereof that can serve as an alternative to fossil-based acrylic acid, acrylic acid derivatives, or mixtures thereof. One of the most valuable application of acrylic acid is that it is used to produce sodium polyacrylate. This polyacrylate is a superabsorbent polymer (SAP) and is used in hygiene products such as diapers. This material can absorb liquids (more than 500 times of its weight).

Currently used diapers are being landfilled and it would be desirable to use such wasted material in a circular economy as feedstock.

For a long time, the dehydration of glycerol and the condensation of formaldehyde with acetaldehyde have been almost the only commercial means of acrolein synthesis. Currently all the acrolein in the world is made from propylene oxidation.

The process for the synthesis of acrylic acid most widely employed industrially uses a catalytic reaction of propylene using an oxygen-comprising mixture. This reaction is generally carried out in the vapour phase and generally in two stages: the first stage carries out the substantially quantitative oxidation of the propylene to give an acrolein-rich mixture in which acrylic acid is a minor component and then the second stage carries out the selective oxidation of the acrolein to give acrylic acid. The reaction conditions of these two stages, carried out in two reactors in series or in the two reaction regions of a single reactor, are different and require catalysts suited to each of the reactions.

CH₂═CH—CH₃+O₂→CH₂═CH—CHO+H₂O stage 1

CH₂═CH—CHO+O₂→CH₂═CH—COOH+N₂O stage 2

Off late, manufacturers have been carrying out research and development studies on processes for the synthesis of acrolein and acrylic acid using bio-resourced starting materials. These studies arise from concern to avoid the use in the future of fossil starting materials, such as propylene, the petroleum origin of which is contributing to global warming due to the greenhouse effect. Furthermore, its cost can only increase in the future with the decline in global oil reserves.

Furthermore, current commercial process that uses aldol condensation approach, including the production of crotonaldehyde (CH₃CH═CHCHO), have many disadvantages due to the high salt content that often results in an unwanted contamination of the final product.

It is thus highly desired to be provided with new means to produce acrylic acid and its derivatives.

### SUMMARY

It is provided a process of converting syngas into acrylic acid comprising converting the syngas into methanol and separating the methanol into a first and second stream; carbonylation of the first stream of methanol producing methyl acetate; hydrolyzing the methyl acetate to obtain acetic acid; and reacting by aldol condensation formaldehyde and the acetic acid to produce acrylic acid.

In an embodiment, the first stream of methanol is dehydrated to produce dimethyl ether (DME) and the DME is further contacted with syngas under an iodide-free environment to produce the methyl acetate by carbonylation. In an embodiment, the H₂/CO ratio is between 0 and 2.

In another embodiment, the carbonylation of methanol and hydrolysis of methyl acetate is conducted in a single catalytic vessel producing acetic acid and dimethyl ether (DME).

In an additional embodiment, the single vessel is a fixed bed reactor.

In another embodiment, the formaldehyde is incorporated following oxidizing of the second stream of the methanol in a gas phase reaction.

In an embodiment, the methyl acetate is hydrolyzed in a reactive distillation process to produce the acetic acid. In a further embodiment, at least 95% or 95-99% carbon based pure acetic acid is produced.

In another embodiment, the methanol is oxidized with excess air at 250-400° C., converting up to 99% of methanol into formaldehyde.

In a further embodiment, the hydrolysis of the methyl acetate is conducted in the presence of methanol to produce the acetic acid.

In an additional embodiment, the carbonylation of the first stream of methanol producing methyl acetate is conducted a gas phase.

In an embodiment, the dehydration of methanol to produce DME is conducted in the presence of a dehydration catalyst.

In a further embodiment, the dehydration catalyst is gamma-alumina.

In an embodiment, the DME is further passed into a packed bed reactor in presence of catalyst to produce the methyl acetate.

In another embodiment, the catalyst is a zeolite or a metal modified zeolite.

In an embodiment, the catalyst comprises a mordenite zeolite, zinc, and copper.

In another embodiment, the unreacted syngas after contacting with DME and producing the methyl acetate is recycled back for conversion of said unreacted syngas into methanol.

In an embodiment, the aldol condensation reaction is conducted in a single-pass, fixed-bed, and flow reactor operating under atmospheric pressure.

In another embodiment, the methyl acetate is hydrolyzed in a reactive distillation column comprising an heterogeneous catalyst.

In another embodiment, the heterogeneous catalyst is an Amberlyst type catalyst.

In an embodiment, the catalyst is activated in presence of air and feed gas mixture.

In an embodiment, the process described herein further comprises a first step of gasifying a carbonaceous material to produce the syngas.

In another embodiment, the carbonaceous material is a liquid, a solid and/or a gas containing carbon.

In a supplemental embodiment, the carbonaceous material is a biomass.

In an embodiment, the carbonaceous material comprises a plastic, a metal, an inorganic salt, an organic compound, industrial wastes, recycling facilities rejects, automobile fluff, municipal solid waste, ICI waste, C&D waste, refuse derived fuel (RDF), solid recovered fuel, sewage sludge, used wood utility poles, wood railroad ties, wood, tire, synthetic textile, carpet, synthetic rubber, materials of fossil fuel origin, expanded polystyrene, poly-film floc, construction wood material, or any combination thereof.

### BRIEF DESCRIPTION OF THE DRAWINGS

Reference will now be made to the accompanying drawings.

FIG. 1 illustrates the non-oxidative process for the manufacture of acrylic acid according to an embodiment.

FIG. 2 illustrates a block flow diagram of the process for the manufacture of acetic acid and DME directly by a reactive distillation (RD) process in accordance to an embodiment.

FIG. 3 illustrates the aldol condensation catalytic test unit used.

FIG. 4 illustrates an experimental equilibrium constant plot showing decrease of methyl acetate hydrolysis as the temperature decreases.

FIG. 5 illustrates the reactive distillation column configuration as encompassed herein.

FIG. 6 illustrates a catalyst basket in accordance to an embodiment which essentially holds the catalyst showing in (a) Amberlyst catalyst (b) SS mesh (c) Catalyst basket.

### DETAILED DESCRIPTION

It is provided the production of acetic acid and acrylic acid and its derivatives from carbon derived materials from waste materials such as industrial waste, municipal solid waste, and biomass.

More particularly, the process described herein includes the production of synthesis gas from carbonaceous materials through a gasification process to produce synthesis gas and utilize the synthesis gas to manufacture acrylic acid. Carbonaceous materials derived from waste resources such as municipal solid waste and biomass are considered renewable and can be used within the existing fossil-fuel infrastructure, that may include coal-fired power plants (co-firing), transportation fuel distribution systems (methanol, dimethyl ether and ethanol) as well as for chemicals production. The gasification process allows synthesis gas production from any waste biomass materials, such as forest residues, agricultural residues, spent structural wood materials, and urban biomass, such as municipal solid waste.

Synthesis gas, also called syngas, is a fuel gas mixture comprising primarily of carbon monoxide (CO), carbon dioxide (CO₂) and hydrogen (H₂). Syngas can be produced from many sources, including biomass, or virtually any carbonaceous material, by reaction with steam (steam reforming), carbon dioxide (dry reforming), air (partial oxidation), oxygen (partial oxidation) or any mixture of the reactants listed.

Carbonaceous material refers to any gas, liquid or solid that contains “carbon” atoms. In most cases, these atoms may be originated from plants or animals and their derivatives, or from fossil fuel and derivatives. Examples of carbonaceous materials include, but are not limited to, Municipal Solid Waste (MSW); Industrial, Commercial, and Institutional waste (IC&I); Construction and Demolition waste (C&D); any petroleum product; plastic; homogenous and/or non-homogeneous biomass.

The carbonaceous materials encompassed herein can be biomass-rich materials which may be gasified in accordance with an embodiment, and include, but are not limited to, homogeneous biomass-rich materials, non-homogeneous biomass-rich materials, heterogeneous biomass-rich materials, and urban biomass. The carbonaceous material can also be plastic rich residues or any waste/product/gas/liquid/solid that include carbon. For example, used diapers are being landfilled and the process described herein allows the use such wasted material as feedstock for the described gasification process as a carbonaceous material; and thus results in a circular diaper usage similar to circular plastic recycling.

In general, urban heterogeneous waste are materials which are obtained from municipal solid waste, such as refuse derived fuel, solid recovered fuel, sewage sludge, putrefied diapers. Gasification of such waste materials are known to those skilled in the art. For example, in a non-limiting embodiment, the biomass may be gasified in a gasifier, which includes a fluidized bed section and a reforming, or freeboard section. Examples of such gasifiers are disclosed in published patents such as U.S. Pat. Nos. 8,080,693, 8,436,215, 8,137,655, 8,192,647, and U.S. Pat. No. 8,636,923 to produce clean syngas.

The carbonaceous materials encompassed herein may also be any type of coal and derivative such as pet coke, petroleum product & by-product, waste oil, oily fuel, hydrocarbon and tar.

Homogeneous biomass-rich materials are biomass-rich materials, which come from a single source. Such materials include, but are not limited to, materials from coniferous trees or deciduous trees of a single species, agricultural materials from a plant of a single species, such as hay, corn, or wheat, or for example, primary sludge from wood pulp, and wood chips. It may also be materials from refined single source like waste cooking oil, lychee fruit bark or stillage from corn to methanol by-product.

Non-homogeneous biomass-rich materials in general are materials, which are obtained from plants of more than one species. Such materials include, but are not limited to, forest residues from mixed species, and tree residues from mixed species obtained from debarking operations or sawmill operations.

Converting carbonaceous materials and waste into synthesis gas can be achieved with gasification techniques. Syngas may be produced by gasifying carbonaceous feedstock. The gasification provides a crude syngas which includes impurities such as ammonia (NH₃), sulfur (as hydrogen sulfide (H₂S) and carbonyl sulfide (COS)), chlorine (as HCl), volatile metals, aromatic tars (NBTX; naphthalene, benzene, toluene and xylene), tars (including HAP), fines ashes (in the form of particles containing metals and metal salts), bed material, and char (solid particulates typically above 0.001 mm and containing metals, salts and mostly carbon). Such impurities, however, limit the ability of the syngas to be used as a fuel or to be employed in the synthesis of other useful materials without a cleaning process.

In an embodiment, it is encompassed an integrated acrylic acid production process entailing formaldehyde and acetic acid synthesis from methanol and syngas, all derived from waste or biomass. In a further embodiment, at least 95% or 95-99% carbon based pure acetic acid is produced. It is provided a process for producing acrylic acid from (a) reacting methanol with excess air at 250-400° C. (methanol conversion up to 99%) to provide a product stream containing formaldehyde; (b) with acetic acid (c) and reacting together in presence of a catalyst to provide a product comprising acrylic acid.

The present disclosure relates to a process and system design for producing acrylates from waste derived methanol. It is also provided the production of acrylic acid and its derivatives from carbon derived from waste materials such as industrial waste, municipal solid waste, and biomass. More particularly, the present disclosure relates to the production of synthesis gas from waste carbon and biomass through gasification process to produce synthesis gas and utilize the synthesis gas to manufacture fuels such as acrylic acid and its derivatives on methanol platform. Both formaldehyde and acetic acid are first derived from methanol (formaldehyde using air oxidation) and acetic acid by using novel application of reactive distillation and iodide free carbonylation. In an alternate embodiment, formaldehyde is provided externally, from an independent feed and thus not derived from methanol. Further all the by-products such as CO₂ are further recycled back into additional syngas through reforming approach. An industrial known commercial catalyst was used and provided acrylic acid with more than 92% selectivity based on formaldehyde conversion of about 50% (formaldehyde is being the limiting agent). Accordingly, the process described herein provides optimized conditions for highest selectivity conversion.

The raw materials, e.g., acetic acid, used in connection with the process described herein are derived from carbonylation of methanol. More specifically the acetic acid could be from methyl acetate hydrolysis in presence of methanol. In another embodiment, the methyl acetate is manufactured on a catalytic route that does not use methyl iodide as a co-catalyst also avoids the use of noble metal such as Rh as carbonylation catalyst. More specifically the alternative source of acetic acid and formaldehyde production could be from waste derived syngas. The intermediates such as methanol and carbon monoxide are manufactured from municipal solid waste or biomass as an alternate carbon source. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid.

In accordance with an embodiment, there is provided a process for producing acrylic acid from a carbonaceous material. The process comprises gasifying the carbonaceous material to provide a crude synthesis gas. The crude synthesis gas then is purified to provide a purified synthesis gas. At least a portion of the carbon monoxide from the purified synthesis gas is reacted with hydrogen from the purified synthesis gas to produce methanol as described in PCT/CA2020/050464, the content of which is incorporated by reference in its entirety. The methanol then is reacted under specified conditions to provide a stream of formaldehyde and in another stream of an intermediate of dimethyl ether (DME). The DME is further contacted with syngas under iodide-free environment to produce methyl acetate. The methyl acetate is subjected to one or more reaction steps to produce acetic acid. The acetic acid and formaldehyde is further contacted under specific reaction condition under a catalyst to produce acrylic acid.

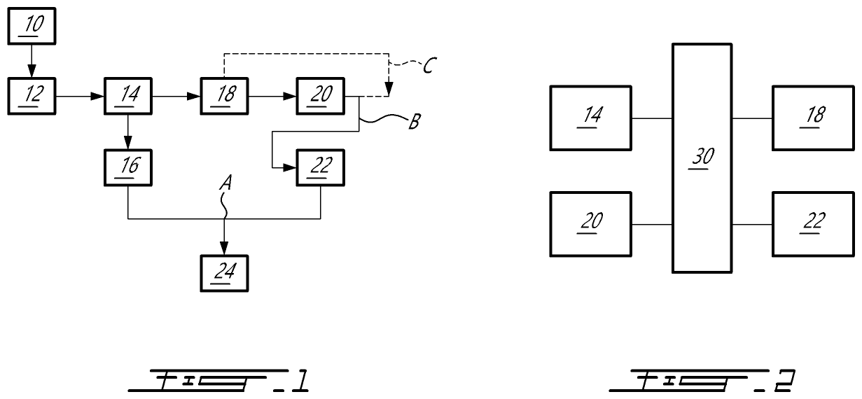

As illustrated in FIG. 1, it is provided a process including gasification of waste materials 10 capable of producing syngas 12 of such composition without importing external hydrogen which improves not only the biogenic content but also reduces the GHG emission significantly. Formaldehyde 16 and acetic acid 22 have been found to participate in an aldol condensation a reaction to form acrylic acid 24. In an embodiment, methanol 14 is produced from waste 10 via synthesis gas 12. An iodide-free carbonylation and reactive distillation step B is used to make acetic acid 22 using dimethyl ether (DME) 18, a methanol derivative, which is then hydrolyzed to produce methyl acetate 20 formed by the iodide-free carbonylation which is hydrolyzed in a reactive distillation process to obtain acetic acid 22.

The aldol condensation route provides an economically feasible process for acrylic acid formation, without dependence on the petrochemical industries and with immense carbon capture potential.

As seen in FIG. 1, it is illustrated, in an embodiment, a simplified representation of the process for the manufacture of formaldehyde 16 from methanol 14. The overall conversion of CH₃OH by Formox™ Process to Formaldehyde is reported between 90-92%. Oxidation only with excess air in the presence of a modified iron-molybdenum-vanadium oxide (e.g. V—P—O type) catalyst at 250-400° C. at atmospheric pressure (methanol conversion=98-99%); commercial catalyst is available from catalyst providers with a catalyst life anywhere between 18-24 months. Also represented in FIG. 1 is a simple block flow diagram of the process for the manufacture of methyl acetate 20 using methanol derived DME 18 without using CH₃I as co-catalyst. The process described herein uses a sequence of process units that convert waste to syngas, clean the syngas, compress the syngas, and then convert the syngas after adjusting the right H₂ to CO ratio to produce the product of interest; 2:1 in the case of methanol or ethanol, 1:1 in the case of acetic acid. In one example, the H₂/CO ratio is between 0 and 2. Further, the catalytic carbonylation in excess of methanol is used in a gas phase with a heterogeneous catalyst instead of commercially practiced liquid phase to produce methyl acetate The methyl acetate 20 can also be produced, conceptually, from the carbonylation of DME 18—no water is then present and the use of methyl iodide as a co catalyst can be eliminated.

In general, the hydrogen and carbon monoxide (as syngas) are reacted to produce methanol according to the following equation:

CO+2H₂ [symbol] CH₃OH

In a non-limiting embodiment, the methanol then is subjected to dehydration to produce at least one ether, such as dimethyl ether, or DME, according to the following equation:

2CH₃OH [symbol] CH₃OCH₃+H₂O

The methanol may be subjected to dehydration to produce dimethyl ether in the presence of a dehydration catalyst. In a non-limiting embodiment, the dehydration catalyst is gamma-alumina.

In a non-limiting embodiment, the hydrogen and carbon monoxide are reacted in the presence of an “integrated” methanol synthesis and dehydration catalyst which may be suspended in an inert oil, such as white mineral oil or Drakeol, into which the hydrogen and carbon monoxide are bubbled. In such an embodiment, the hydrogen and carbon monoxide are reacted in the presence of the “integrated” catalyst to produce methanol. The methanol then is reacted immediately in the presence of the “integrated” catalyst to produce DME and water. In a non-limiting embodiment, the hydrogen and the carbon monoxide are reacted in the presence of a methanol catalyst in a first reactor to produce methanol, and then the methanol is reacted in the presence of a dehydration catalyst in a second reactor to produce at least one ether, such as DME.

The DME then is purified to remove the residual hydrogen, carbon monoxide and water. The purified DME then is passed to a reactor such as, for example, in a non-limiting embodiment, a packed bed reactor in presence of catalyst such as zeolite or metal modified zeolite to produce selectively methyl acetate. Examples of such is seen in U.S. Pat. No. 10,695,756 which relates to catalysts used in the conversion of dimethyl ether to methyl acetate, in which dimethyl ether is reacted with carbon monoxide to produce methyl acetate. More particularly, encompassed are catalysts used in the conversion of dimethyl ether to methyl acetate (MA), wherein the catalyst comprises (i) a mordenite zeolite; (ii) zinc; and (iii) copper, wherein said copper and said zinc are present in said catalyst at a molar ratio of said copper to said zinc of about 0.25. Depending upon the catalyst used for the MA synthesis, the selectivity may be between 80% and 95%.

Aldol condensation reactions of carbonyl compounds can occur on the catalyst active sites both basic and acidic types. Both basic and acidic type catalysts are used presently. In most cases, the use of basic type catalysts is characterized by the satisfactory selectivity of AA formation, however the conversion rate of the limiting reactor on catalysts are relatively low. On the contrary, acid type catalysts provide higher conversion of the reactants however, their use is accompanied by formation of a large quantity of by-products.

The aldol condensation reaction can be catalyzed by acid, base and acid-base bi-functional catalysts including alkaline earth metal oxides such as magnesium oxide or calcium oxide, alkali promoted alkaline earth metal oxides such as lithium, sodium, potassium or cesium promoted magnesium oxide, supported alkali catalysts, acidic zeolites, alkali modified zeolites, magnesium-aluminum hydrotalcites, anionic clay, zirconia, sulfate modified zirconia, lanthanum oxide, niobium oxide, cerium oxide, titanium oxide.

As seen in FIG. 1 and provided herein, the process uses the aldol condensation of acetic acid and formaldehyde both traditionally based on methanol platform. The iodide-free carbonylation process described herein produces methyl acetate and utilizes direct syngas and DME. Alternatively, as encompassed herein a process intensification approach for reactive distillation that utilizes methanol and methyl acetate produces DME and acetic acid. In an embodiment, all or part of the syngas can be recycled.

The overall stoichiometry's, related to initial syngas, and is different for these two routes:

### Via Propylene Oxidation:

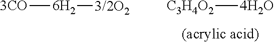

The required synthesis gas ratio H₂/CO=2/1.

Via Aldol Condensation Route:

3CO-4H₂-½O₂C₃H₄O₂-2H₂O

The required synthesis gas ratio H₂/CO=4/3.

Thus, the route via aldol condensation appears more efficient since it requires less hydrogen per unit of CO (1.34 vs 2). The gasification step used herein is capable of producing syngas of such composition without importing the external hydrogen which improves not only the biogenic content but also reduces the GHG emission significantly. The aldol condensation route used herein provides a means to deliver an economically feasible process for acrylic acid formation, without dependence on the petrochemical industries. An integrated waste derived syngas process based on non-oxidative approach allows manufacturing of acrylic acid without dependence on the fossil fuel industry and with immense carbon capture potential.

It was demonstrated that the vapor-phase aldol condensation reaction as proposed herein is performed in a single-pass, fixed-bed, and flow reactor operating under atmospheric pressure. The reaction temperature was in the range of 623-693 K and the mixed feed of acetic acid and formaldehyde (different concentration from 25-37% in methanol and water) with ratio in the range of 1-10 has been tested on a variety of catalyst. Depending on the limiting reagent a selectivity of more than 90% (by mole) has been obtained for acrylic acid.

The process described for the manufacture of acrylic acid from acetic acid and formaldehyde comprises at least the following stages: waste derived methanol is subjected to oxidation in presence of excess air and a commercially available catalyst at 250-400° C. at atmospheric pressure (methanol conversion=98-99%); commercial catalyst is available from Johnson Matthey; Catalyst Life=18-24 months. Overall conversion of methanol by Formox™ Process to Formaldehyde—86% (90-92%) reported.

Carbonylation is also done commercially in gaseous phase using Rh catalyst in presence of methyl iodide which, under appropriate conditions that provide acetic acid and associated products (U.S. Pat. No. 8,080,693). If the reaction of methanol and carbon monoxide is conducted under conditions having a sufficient molar ratio of methanol to carbon monoxide, i.e., a sufficient equimolar of methanol vis-a-vis carbon monoxide, and a sufficient acidity the catalytic carbonylation results into acetic acid. The molar excess of methanol compared to CO could also lead to esterification to methyl acetate, however, the molar ratio of methyl acetate to acetic acid in the reaction product is a result of the kinetic rate of the acid catalysis following the carbonylation reaction, and it is limited by the equilibrium between the reactants and products. The equilibrium between reactants and products may be altered by changing reaction conditions such as temperature, pressure, and composition of reactants.

Enerkem Inc. for example is known to conduct carbonylation in a vapor/gas phase catalytic flow reactor in excess of methanol as reactant (with respect CO) to obtain methyl acetate (CH₃COOCH₃, MA), as main product. MA is a well-known solvent and is also used in organic synthesis including acetic acid. The main drawback of the current process is that it requires CH₃I, which inevitably forms HI that is highly corrosive and also requires full recovery (>99.99%) downstream due to its toxicity. This translates into significant capital and operational expenses. It is thus particularly disclosed a process of producing acetic acid by using an auxiliary reaction to methyl acetate hydrolysis by a reactive distillation process. This allows in particular to accomplish an iodide free gas phase carbonylation of dimethyl ether (DME) to acetic acid.

As provided herein, methyl acetate (MA) is manufactured in an iodide-free process and it follows that the manufacture of acetic acid is also accomplished iodide free. The reaction requires H₂/CO ratio of 1/1 as depicted below for acetic acid synthesis:

CO+2H₂<=>CH₃OH

2CH₃OH<=>CH₃OCH₃+H₂O

CH₃OCH3+CO<=>CH₃COOCH₃(MA)

CH₃COOCH₃+H₂O<=>CH₃COOH+CH₃OH

2CO+2H₂═CH₃COOH

where CO₂ can be dry-reformed with CH₄ to produce 1/1 CO/H₂, and methanol can be dehydrated to DME.

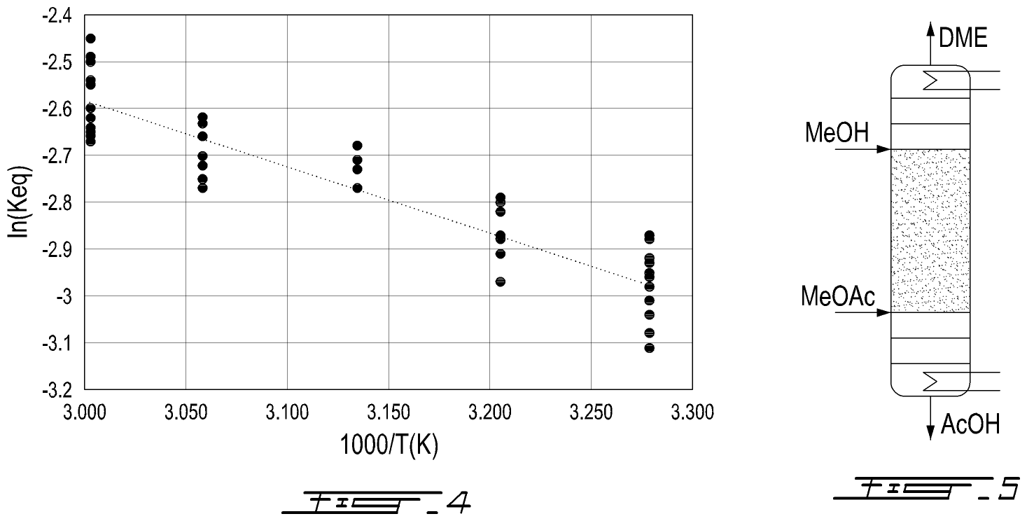

There is a significant knowledge accumulated on MA hydrolysis in polymer industry. Polyvinyl alcohol (CH₂═CH—OH)ₙ, PVA) is an important material for producing synthetic fiber, film etc. In the process PVA synthesis, methyl acetate is produced as a by-product with a high yield. It is estimated that 1.5-1.7 ton of MA is produced per ton of PVA. MA is usually hydrolyzed to methanol and acetic acid and are recycled to the methanolysis reaction of polyvinyl acetate and the synthesis of vinyl acetate monomer (VAM) respectively. The hydrolysis reaction is carried out in the fixed-bed reactor catalyzed by ion exchange resin. Limited with a equilibrium constant ˜0.14 at 25° C., the hydrolysis ratio is relatively low (˜23%), resulting in a large amount of recirculation resulting in a significant increase in energy. It can be seen in FIG. 4 how experimental chemical equilibrium constants (K_{eq}) of methyl acetate hydrolysis decreases as the temperature decreases.

Furthermore, in order to deal with the azeotropes of methyl acetate-methanol and methyl acetate-water existing in the system, a complex separation process is required and up to 4 separation columns are normally used.

As tested and completed on two different types of solid acid catalysts such as Mordenite (H-MOR) and gamma-alumina, it also indicates that under H₂O/MA ratio of 20 high conversion of MA is achievable at relatively milder temperature (100° C.). Based on the experiments conducted it is unlikely that the MA conversion can be improved or energy consumption reduced by reducing the molar feed ratio of H₂O/MA.

A number of improved processes have been developed to overcome the disadvantages mentioned. It is provided that reactive distillation, a process that combines reaction and separation together, is an attractive alternative process and it gives clear advantages for systems with small equilibrium constant.

The existence of binary azeotropes is thus observed: (1) methyl acetate and methanol form a minimum-boiling azeotrope with the composition of 65.9 mol % methyl acetate at 53.7° C., and (2) methyl acetate and water forms minimum-boiling azeotrope with the composition of 89.0 mol % at 56.4° C. Both are predicted at atmospheric pressure. Thus, the order of the normal boiling point temperature for pure components and azeotropes is:
HOAc>H₂O>MeOH>MeOAc>MeOAc/H₂O>MeOAc/MeOH
118° C.>100° C.>64.5° C.>57.5° C.>56.4° C.>53.6° C.

When considering a reaction depicted as A+B=C+D, where the boiling points of the components follow the sequence A>B>D>C. The traditional flow-sheet for this process consists of a reactor followed by a sequence of distillation columns
A is acetic acid (118)
B is methanol (64.5)
C is Methyl acetate (57.5)
D is water (100)

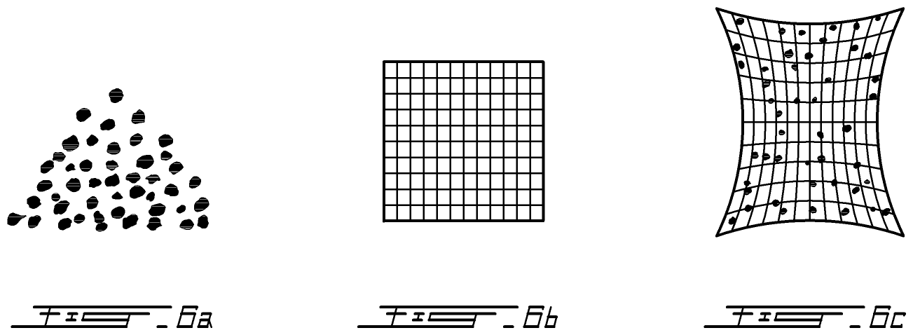

The most spectacular example of the benefits of RD is in the production of methyl acetate. The acid catalysed reaction MeOH+MeOAc=DME+AcOH was traditionally carried out using one reactor and a train of nine distillation columns. As proposed herewith, the RD implementation (see FIG. 5) only one column is required and nearly 100% conversion of the reactant is achieved. The capital and operating costs are significantly reduced.

Based on the above, an acetic acid synthesis that use H₂/CO ratio of 1 is provided.

It is encompassed that the most efficient route for the production of acrylic acid is one having effective H/C ratios (H/C_{eff}) which is as close to zero as possible for all compounds involved in the production route. H/C_{eff} is defined as follows, based on the carbon content (C), hydrogen content (H) and oxygen content (O) of the compound in question (expressed as atomic ratio):

H/C_{eff}=(H−2*O)/C.

For illustration purposes, this definition when applied to CH₄ results in H/C_{eff}=4. When applied to CO₂, it results in the opposite: H/C_{eff}=−4. It was surprisingly noticed that for acrylic acid (CH₂CHCOOH) have H/C_{eff}=0. In contrast, propylene (CH₂═CH—CH₃) is characterized by H/C_{eff}=2. On the other hand, both formaldehyde (HCHO) and acetic acid (CH₃COOH) are favorably characterized by a H/C_{eff} which is zero and represent therefore a more efficient feedstock for or intermediate in the production of acrylic acid as presented in the disclosure.

In accordance with an embodiment, there is provided a process for producing acrylic acid from a carbonaceous material. The process comprises gasifying the carbonaceous material to provide a crude synthesis gas. The crude synthesis gas then is purified to provide a purified synthesis gas. At least a portion of the carbon monoxide from the purified synthesis gas is reacted with hydrogen from the purified synthesis gas to produce methanol as described in PCT/CA2020/050464, the content of which is incorporated by reference in its entirety. The methanol then is reacted under specified conditions to provide a stream of dimethyl ether (DME). The DME is further contacted with syngas under iodide-free environment to produce methyl acetate.

As particularly encompassed in FIG. 1, it is provided a process wherein methanol 14, produced for example from waste via synthesis gas is used to make acetic acid 22. An iodide-free carbonylation and reactive distillation step B is used to make acetic acid 22 using dimethyl ether (DME) 18, a methanol derivative, which is then hydrolyzed to produce methyl acetate 20 formed by the iodide-free carbonylation which is hydrolyzed in a reactive distillation process to obtain acetic acid 22.

In an embodiment, methanol is produced from waste via synthesis gas. The process encompass the manufacture of methyl acetate using methanol derived DME without using CH₃I as co-catalyst. The process described herein uses a sequence of process units that convert waste to syngas, clean the syngas, compress the syngas, and then convert the syngas after adjusting the right H₂ to CO ratio to produce the product of interest, 1:1 in the case of acetic acid. Further, the catalytic carbonylation in excess of methanol is used in a gas phase with a heterogeneous catalyst instead of commercially practiced liquid phase to produce methyl acetate.

In general, the hydrogen and carbon monoxide (as syngas) are reacted to produce methanol according to the following equation:

CO+2H₂ [symbol] CH₃OH

In a non-limiting embodiment, the methanol then is subjected to dehydration to produce at least one ether, such as dimethyl ether, or DME, according to the following equation:

2CH₃OH [symbol] CH₃OCH₂+H₂O

Particularly, as seen in FIGS. 1 (step C) and 2, it is described a process for the manufacture of acetic acid 22 and DME 18 directly by a reactive distillation (RD) process 30 intensified by an auxiliary reaction. The present process is based on the chemical equilibrium and kinetically controlled design in a novel process that hydrolyze MeOAc and MeOH in the integrated process of acetic acid manufacturing. Compared to traditional processes, no additional water is required to feed into the RD column and the process is significantly simplified. In another embodiment of the process with equal mole of MeOAc and MeOH as feed coupled with a set of pre-reactor close to 100% conversion of MeOAc and MeOH is achieved with a high purity dimethyl ether and acetic acid as products in the RD column.

The described reactive distillation allows the simultaneous reaction (dehydration of methanol and hydrolysis of methyl acetate) in a single catalytic vessel to manufacture acetic acid and DME and their separation by difference in boiling point without forming any azeotrope is applied.

Processes for the manufacture of acetic acid from methanol by carbonylation are operated extensively (see Howard et al. in Catalysis Today, 18 (1993) 325-354). All commercial processes for the preparation of acetic acid by the carbonylation of methanol presently are performed in the liquid phase using homogeneous catalyst systems comprising a Group VIII metal and iodine or an iodine-containing compound such as hydrogen iodide and/or methyl iodide. Rhodium is the most common Group VIII metal and methyl iodide is the most common promoter. These reactions are conducted in the presence of water to prevent precipitation of the catalyst.

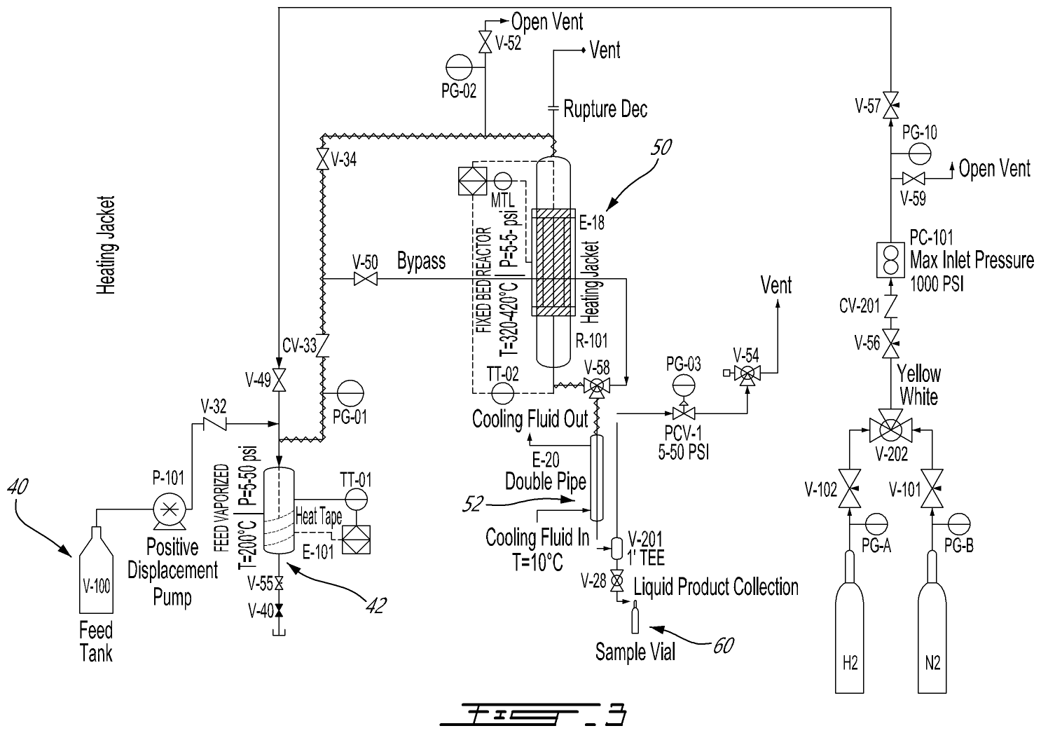

In a non-limiting embodiment, the methyl acetate hydrolysis could also results into acetic acid as represented in FIG. 2. This is a conceptual implementation of the process herein to manufacture acetic acid as an attractive alternative in the form of reactive distillation (RD) for process intensification with the advantages of simultaneous reaction and separation. The hydrolysis of methyl acetate is often performed in a fixed bed reactor followed by several separation steps including distillation and extractive distillation. However, the yield of a fixed bed reactor is limited by the equilibrium of the chemical reaction, meaning that the conversion of methyl acetate to acetic acid and methanol will be completed only to the equilibrium point. Due to the fact that a substantial amount of methyl acetate and water remain unreacted in the fixed bed reactor as a consequence, large recycle streams have to be provided.

As seen in FIG. 2, in an alternative embodiment, the simple block flow diagram of the process for the manufacture of acetic acid 22 and DME 18 directly by a reactive distillation (RD) process 30 intensified by an auxiliary reaction is implemented. The present process is based on the chemical equilibrium and kinetically controlled design in a novel process that hydrolyze MeOAc and MeOH in the integrated process of acetic acid manufacturing. Compared to traditional processes, no additional water is required to feed into the RD column and the process is significantly simplified. In another embodiment of the process with equal mole of MeOAc and MeOH as feed coupled with a set of pre-reactor close to 100% conversion of MeOAc and MeOH is achieved with a high purity dimethyl ether and acetic acid as products in the RD column.

Thus it is encompassed a process that applies RD configuration to obtain acetic acid using an auxiliary reaction as per the following reactions:

CH₃₂COOCH₃+H₂O [symbol] CH₃OH+CH₂COOH;ΔH₂₉₈°=10.87 kJ/mol

2CH₃OH [symbol] CH₃OCH₃+H₂O;ΔH₂₉₈°=−35.27 kJ/mol

### Overall Reaction

CH₃COOCH₃+CH₂OH [symbol] CH₃OCH₃+CH₂COOH

Since RD is a system which allows to have simultaneous reaction and product separation it is more energy efficient with significant capital and operating cost reduction and most importantly higher conversion via efficient product separation with enhanced reaction equilibrium conditions by providing a distillation column, in which a reaction section comprising structured packing for performing the reaction of methanol and methyl acetate to DME and acetic acid. The reaction section of the column, in which the chemical reaction takes place, contains a heterogeneous catalyst, such as Amberlyst type catalyst under optimized temperature and flow rate. One of the ideal process configurations of RD consists of a column where the light and heavy reactants are fed at the lower and upper parts of the reactive zone while the heavy and light components are bottom and top products, respectively.

During the reaction and separation process in the reactive distillation, it is still possible MeOAc+MeOH goes up as light components and water goes down as heavy component. Hence, the reactants contact does not happen with the catalyst. Therefore, without wising to be bound by theory, a certain split flow of the top condenser liquid could be pumped around at lower stage of the distillation unit to have better contact with catalyst. Essentially this allows to achieve higher reactant conversion. The experimental results were validated via ASPEN-Hysys simulation, and was implemented in simulation to demonstrate the MeOAc+MeOH pump around effectiveness to augment the conversion significantly.

Once the contact of reactant occurs effectively with the heterogeneous catalyst, the reaction progresses. In addition to that, separation of products enhance the reaction to forward direction. However, if the reaction zone contains only catalyst without basket, the pressure drop problem occurs. The pressure increment at the bottom reaction zone increases the boiling point of the product. This essentially creates flooding problem at the bottom part of the distillation unit. Eventually the distillation system gets unstable and the separation does not happen effectively.

The catalysts may be placed in reactive distillation using a specially designed catalyst basket (see FIG. 6). The basket can help to avoid potential problems due to the pressure drop of the catalyst bed. Generally, the pressure drop is primarily caused by the resistance due to a small size catalyst. The catalyst basket as seen in FIG. 6c can be designed to be performed as packing material at the reaction zone. This enhances the reaction performances as well as the product separation efficiency. Accordingly, in some embodiments, the Amberlyst type catalyst (see FIG. 6a) is wrapped by a SS mesh (see FIG. 6b) creating a catalyst basket (FIG. 6c). The catalyst basket avoids this operational problems (e.g. pressure drop) as discussed in paragraph hereinabove. In addition to that, the catalyst baskets create large surface area to have more equilibrium stages for products separation according to their boiling point. Product removal essentially progresses the reaction equilibrium to forward direction and enhances the reaction performance. The indirect hydration of cyclohexene to cyclohexanol using a reactive entrainer is a typical example of intensification (Steyer et al., 2008, Ind. Eng. Chem. Res. 47, 9581 and Katariya, et al., 2009. Two-step reactive distillation process for cyclohexanol production from cyclohexene. Ind. Eng. Chem. Res. 48, 9534). As the liquid-liquid phase splitting in the system of cyclohexene/water limits the reaction rate, formic acid as the reactive entrainer is introduced into the system. Cyclohexene first reacts with formic acid forming formic acid cyclohexyl ester, and then this ester can be split with water into cyclohexanol and formic acid. The reaction route offers the advantage of overcoming reaction rate limits and having no significant amounts of side products. Similar concept is applied in one of the aspects described herein where a novel process of hydrolysis of MeOAc is developed based on the equilibrium and kinetically controlled design. Compared with conventional processes, the novel process can be significantly simplified with 94% conversion of MeOAc with acetic acid purity at reboiler with up to 99% has been proven. The catalytic experiments were carried out using a glass distillation where catalysts were held using catalyst basket. The operation conditions were T=80-100° C., and a pressure close to atmosphere. Compared to the glass unit, the stainless steel (SS) reactive distillation was at least a 5 times larger unit. A MeOAc conversion rate of 40-94% was achieved depending on the operation condition and reactor configuration. The carbon-based purity level of acetic acid was 95-99 mol %. Furthermore, high purity dimethyl ether (DME) as an additional product as also obtained through methanol dehydration.

Formaldehyde and acetic acid have been found to participate in an aldol condensation reaction to form acrylic acid. The aldol condensation route provides a mean of delivering an economically feasible process for acrylic acid formation, without dependence on the petrochemical industries. The current process can be designed to produce acetic acid in the carbonylation rector and reactor dynamics can be changed so that acetic acid is the major product. The process was described in a paper by Vitcha and Sims (Vapor Phase Aldol Reaction. Acrylic Acid by Reaction of Acetic Acid and Formaldehyde. Industrial & Engineering Chemistry Product Research and Development 1966, 5 (1), 50-53). Subsequently a variety of patents such as U.S. Pat. Nos. 3,840,587, 4,339,598, 4,165,438, 8,507,721, and 9,120,743 disclose the processes for preparing acrylic acid from methanol and acetic acid in which the methanol is partially oxidized to formaldehyde in a heterogeneously catalyzed gas phase reaction. The acetic acid is used in excess over the formaldehyde. The formaldehyde present in reaction gas input mixture is aldol condensed with the acetic acid via heterogeneous catalysis to form acrylic acid. Unconverted acetic acid still present alongside the acrylic acid in the product gas mixture is removed therefrom and is recycled to the reaction gas input mixture.

Aldol condensation reactions of formaldehyde and acetic acid and/or carboxylic acid esters are described in U.S. Pat. No. 8,507,721 where the reaction is carried out over a catalyst and forms acrylic acid.

U.S. Pat. No. 9,695,099 discloses a process for preparing acrylic acid from methanol and acetic acid in which the methanol is partially oxidized to formaldehyde in a heterogeneously catalyzed gas phase reaction. The product gas mixture thus obtained, and an acetic acid source are used to obtain a reaction gas input mixture that comprises acetic acid and formaldehyde. The acetic acid is used in excess over the formaldehyde. The formaldehyde present in reaction gas input mixture is aldol-condensed with the acetic acid via heterogeneous catalysis to form acrylic acid. Unconverted acetic acid still present alongside the acrylic acid in the product gas mixture is removed therefrom and is recycled to the reaction gas input mixture.

The aldol condensation reaction of the acetic acid with formaldehyde results in the formation of acrylic acid. This reaction has been observed to take place in the region of 280-400° C. and is slightly exothermic.

HCHO+CH₃COOH→CH₂(OH)CH₂COOH→CH₂═CHCOOH+H₂O

ΔH_{f}°=−23.43 kJ/mol (1)

There are however a variety of side reactions possible in the system as well

Esterification of acetic acid with methanol from formalin results in the formation of methyl acetate, the reaction of which with formaldehyde can lead to the formation of methyl acrylate.

CH₃OH+CH₃COOH→CH₃COOCH₃+H₂O (2)

CH₃COOCH₃+HCHO→CH₂═CHCOOCH₃+H₂O (3)

Alternatively, the methanol can also react with acrylic acid directly to form the methyl acrylate.

CH₃OH+CH₂═CHCOOH→CH₂═CHCOOCH₃+H₂O

Carbon dioxide and acetone are formed by the decomposition of acetic acid.

2CH₃COOH→CH₃COCH₃+CO₂+H₂O (4)

The decomposition of formaldehyde can also result in formation of methanol and carbon dioxide as well.

2HCHO→HCOOCH₃→CH₃OH+HCOOH→CH₃OH+CO₂+H₂O (5)

Furthermore, the acrylic acid produced in the system may undergo polymerization to form polyacrylates.

nCH₂═CHCOOH→[—CH₂—CH(COOH)—]ₙ (6)

The process provided herewith allows production of unsaturated acids, e.g., acrylic acids, or esters thereof (alkyl acrylates), by contacting an alkanoic acid with, a methyleneating agent, under conditions effective to produce the unsaturated acid and/or acrylate. Preferably, acetic acid is reacted with formaldehyde in the presence of a catalyst.

The raw materials, e.g., acetic acid, used in connection with the process described herein are derived from carbonylation of methanol. More specifically the acetic acid could be from a non-traditional route such as methyl acetate hydrolysis in presence of methanol. In another embodiment, the methyl acetate is manufactured on a catalytic route that does not use methyl iodide as a co-catalyst when using Rh carbonyls as a carbonylation catalyst.

More specifically the alternative source of acetic acid and formaldehyde production could be from waste derived syngas. The intermediates such as methanol and carbon monoxide are manufactured from municipal solid waste or biomass as an alternate carbon source. By retrofitting a methanol plant, the large capital costs associated with CO generation for a new acetic acid plant are significantly reduced or largely eliminated. All or part of the syngas is diverted from the methanol synthesis loop and supplied to a separator unit to recover CO, which is then used to produce acetic acid. Examples of biomass include, but are not limited to, agricultural wastes, forest products, grasses, and other cellulosic material, wood pellets, cardboard, paper, plastic, and other commercial waste containing carbon.

### Example I

### Aldol Condensation Reaction

Regarding the aldol condensation reaction, the experimental setup used consisted of a jacketed reactor 50 of diameter of 1″ nominal diameter with a chilled collector tank 60 downstream for product collection (see FIG. 3). The liquid feed mixture from the feed tank 40 was pumped into a preheating unit (200° C.) at pre-determined rate 0.2 to 1 ml/min under N₂ flow to carry over the feed vapors through the evaporator 42 into the reactor 50 that contain the catalyst. The vapor-phase aldol condensation reaction was performed in a single-pass, fixed-bed, and flow reactor 50 operating under atmospheric pressure.

The catalyst sample was placed in the middle of the reactor 50, and quartz sands were used both under and above the catalyst sample. The catalysts were crushed and sieved to 50-35 mesh (300 μm to 500 μm) for activity evaluation. The amount of catalyst was from 5.0 gram to 10.0 g and it was doped with the quartz sands to a constant volume of 76 ml. Further, the space above the catalyst bed was filled with α-Al₂O₃ bead of 0.8 mm size to preheat the in-coming liquid from evaporator unit 42. The reaction temperature was in the range of 623-693 K. and the space above the catalyst bed was filled with quartz chips to preheat the in-coming liquid. The feed evaporator unit 42 consisted of a 300 ml stainless steel sample cylinder with a closed-ended and perforated dip-tube. The sample cylinder heated with a heating tape. A thermocouple was placed inside the dip-tube with its tip at the center to control the heating rate. The sample cylinder was filled with quartz to facilitate temperature minimize temperature lags between the center of the cylinder to the sides. The N₂ gas as well as the liquid feed was pumped into the line to the feed tube. The vaporized feed was carried to the reactor system 50. A condenser 52 and a collector tank 60 were placed downstream of the reactor for product collection.

Residence time was optimized (from 5 s to 25 s). The operating temperatures were varied from 350 to 420° C. A variety of formaldehyde solution with varying concentration (25 wt. % to 37 wt. %) were used in addition to trioxane. The mixed solution of acetic acid and formaldehyde was introduced into the reactor 50 via a pre-heater by an HPLC pump with a feed rate from 0.01 to 0.1 ml/min. The molar ratio of acetic acid and formaldehyde was dependent on the catalyst used and varies from either less than % or more than %. Products from the reactor 50 pass through the condenser 52 operated at 4° C. Liquids are collected at the bottom of the condenser and gases pass through an activated carbon adsorbent before being released to the atmosphere.

The effluent products of the liquid phase and the gas phase were analyzed using an Agilent 7820A Gas Chromatography system with FID and DB Wax column and a HaySep column coupled with TCD were utilized, respectively.

The water content in the product was determined by using a Mettler Toledo V20 Volumetric Karl Fischer Titrator. The formaldehyde content in the product samples were determined by titration using the sodium sulphite method. Depending on the concentration of the limiting agent present in the feed mix; conversion, selectivity and yields are defined as per the following Equations while taking formaldehyde as limiting agent:

Conversion: (Moles of Formaldehyde_{IN}−Moles of Formaldehyde_{OUT})×100/Moles of Formaldehyde_{IN}

Selectivity: Moles of Acrylic acid formed×100/Moles of Formaldehyde consumed

Yields: Moles of Acrylic Acid formed×100/Moles of Formaldehyde_{IN}

The vanadyl pyrophosphate (VPO) catalysts are used for the selective oxidation of light alkanes, which are based on vanadyl hydrogen phosphate hemihydrate (VOHPO₄·0.5H₂O) as the precursor. The VPO catalyst is a commercially available product (SynDane® catalyst from Clariant) however, it is widely used in partial oxidation of n-butane to maleic anhydride.

VPO type catalysts have also been employed for conversion of acetic acid and formaldehyde to acrylic acid via a condensation route. Even though this catalyst system has been subjected to laboratory and industrial efforts, many details remain unknown. An appropriate activation procedure can be used to make such VPO catalysts to functionalize aldol condensation.

As encompassed herein, both commercial Syndane catalyst and an in-house modified version of VPO catalyst are able to achieve acrylic acid selectivity (based on formaldehyde) of 80-90% and formaldehyde conversion of 40-50%.

It has been discovered that the catalysts can be activated in presence of air and feed gas mixture to obtain higher selectivity to acrylic acid. It is discovered that the catalysts should remain under oxidized state while under activation. The following activation procedure was applied:
11—Set the temperature to 350° C. for catalyst calcination under N₂ for 1 hour
2—Increase the temperature to 500° C. under N₂ for 1 hour
3—Switch to air at 500° C. under for 2 hour
4—Lower the temperature to 350° C. under air for 2 hour (pre-heater valve is closed)
5—Start the HPLC pump set at (0.001 to 0.1 mL/min with N₂ flow from 10-70 mL/min for 1 hour (pre-heater valve is open)
6—Lower the N₂ gas flow from 10 to 30 mL/min for 30 minutes
7—Switch to air at 30 mL/min for from 10 to 30 minutes
8—Increase the flow of air to 50 to 90 mL/min
9—Activation continue under feed-mix concentration and air for 1 hour
10—Switch to N2 from 50 to 90 mL/min after 1 hour under feed-mix
11—Activation is completed.

### Example II

### Aldol Condensation Catalyst Syntheses

The VPO precursor was prepared by employing the reaction of vanadium pentoxide (32.9 g) with isobutanol (120 ml) in benzyl alcohol (120 ml). The reaction mixture was refluxed for 5 hours at 140° C. for 5 hours. A calculated amount of PEG 6000 was added to the above mix. After 1-hour, phosphoric acid was added slowly to obtain the P/V ratio of 1.05 and refluxed for another 6 h. The turbid reaction mixture was filtered, and the obtained bluish greenish precipitate was oven dried at 120° C. A vanadyl hydrogen phosphate hemihydrate phase (VOHPO₄·0.5H₂O) was obtained which was confirmed by the XRD analysis.

The catalysts precursor (VOHPO₄·0.5H₂O) was loaded on TiO₂ using dry impregnation method. Then activated at different conditions to respectively obtain γ-VOPO₄/TiO₂ and δ-VOPO₄/TiO₂. The activation of the catalysts was carried out in the reactor. An amount of 5.0 g of VPO/TiO₂ was loaded to the reactor. The catalysts precursor was activated at 400° C. for 9 h under air to yield δ-VOPO₄/TiO₂ phase. The precursor was activated at 680° C. for 10 h to yield γ-VOPO₄/TiO₂. Later, γ-VOPO₄/TiO₂ and δ-VOPO₄/TiO₂ with a mass ratio of 1:3 were subjected to solid-solid wetting.

While the disclosure has been described in connection with specific embodiments thereof, it will be understood that it is capable of further modifications and this application is intended to cover any variations, uses, or adaptations and including such departures from the present disclosure as come within known or customary practice within the art to which and as may be applied to the essential features hereinbefore set forth, and as follows in the scope of the appended claims.

What is claimed is:

1. A process of converting syngas into acrylic acid comprising:
a) converting said syngas into methanol and separating the methanol into a first stream;
b) carbonylation of the first stream of methanol producing methyl acetate;
c) hydrolyzing the methyl acetate to obtain acetic acid; and
d) reacting by aldol condensation formaldehyde and the acetic acid produced in c) to produce acrylic acid;
wherein the carbonylation of methanol and hydrolysis of methyl acetate is conducted in a single catalytic vessel producing acetic acid and dimethyl ether (DME).

2. The process of claim 1, wherein the single vessel is a fixed bed reactor.

3. The process of claim 1, wherein in step a) the methanol is separated into a second stream, and the formaldehyde is incorporated following oxidizing of the second stream of the methanol in a gas phase reaction.

4. The process of claim 1, wherein a H₂/CO ratio is between 0 and 2.

5. The process of claim 1, wherein the methyl acetate is hydrolyzed in a reactive distillation process to produce the acetic acid.

6. The process of claim 5, wherein at least 95% to 99% of carbon based pure acetic acid is produced.

7. The process of claim 3, wherein the methanol of the second stream is oxidized with excess air at 250-400° C., converting up to 99% of methanol into formaldehyde.

8. The process of claim 1, wherein the hydrolysis of the methyl acetate is conducted in the presence of methanol to produce the acetic acid.

9. The process of claim 1, wherein the carbonylation of the first stream of methanol producing methyl acetate is conducted a gas phase.

10. The process of claim 1, wherein the aldol condensation reaction is conducted in a single-pass, fixed-bed, and flow reactor operating under atmospheric pressure on a vanadyl pyrophosphate (VPO) catalyst.

11. The process of claim 3, wherein the methyl acetate is hydrolyzed in a reactive distillation column comprising a heterogeneous catalyst.

12. The process of claim 11, wherein the heterogeneous catalyst is an Amberlyst type catalyst.

13. The process of claim 12, wherein the Amberlyst type catalyst is in a mesh forming a catalyst basket.

14. The process of claim 7, wherein the catalyst is activated in presence of air and feed gas mixture.

15. The process of claim 1, further comprising a first step of gasifying a carbonaceous material to produce the syngas.

16. The process of claim 15, wherein the carbonaceous material is a liquid, a solid and/or a gas containing carbon.

17. The process of claim 15, wherein the carbonaceous material is a biomass.

18. The process of claim 16, wherein the carbonaceous material comprises a plastic, a metal, an inorganic salt, an organic compound, industrial wastes, recycling facilities rejects, automobile fluff, municipal solid waste, Industrial, Commercial, and Institutional waste (IC&I), Construction and Demolition waste (C&D), refuse derived fuel (RDF), solid recovered fuel, sewage sludge, used wood utility poles, wood railroad ties, wood, tire, synthetic textile, carpet, synthetic rubber, materials of fossil fuel origin, expanded polystyrene, poly-film floc, construction wood material, or any combination thereof.

19. A process of converting a carbonaceous material into acetic acid using a reactive distillation column comprising:
a) carbonylation of the carbonaceous material in gas phase producing dimethyl ether (DME) in an iodide-free environment;
b) hydrolyzing the DME to produce methyl acetate; and
c) hydrolyzing the methyl acetate to produce acetic acid,
wherein the hydrolysis of the DME and subsequently of the methyl acetate is done in the reactive distillation column, and wherein the H₂ to CO ratio of the carbonaceous material to produce the acetic acid is of 1:1.

20. The process of claim 19, wherein the acetic acid and DME produced in the reactive distillation column are separated by difference in boiling point.

21. (canceled)
